(19) United States
(12) Patent Application Publication
(10) Pub. No.: US 2024/0115537 A1
(43) Pub. Date: Apr. 11, 2024

(54) COMPOSITION FOR PREVENTING OR TREATING BREAST CANCER COMPRISING COMPOUND DERIVED FROM DENDROPANAX MORBIFERUS

(71) Applicant: JEJU NATIONAL UNIVERSITY INDUSTRY-ACADEMIC COOPERATION FOUNDATION, Jeju-si, Jeju-do (KR)

(72) Inventors: Dong-Sun LEE, Jeju-si (KR); Hack-Sun Choi, Jeju-si (KR)

(73) Assignee: JEJU NATIONAL UNIVERSITY INDUSTRY-ACADEMIC COOPERATION FOUNDATION, Jeju-si, Jeju-do (KR)

(21) Appl. No.: 18/321,965

(22) Filed: May 23, 2023

(30) Foreign Application Priority Data
Sep. 23, 2022 (KR) 10-2022-0120436

(51) Int. Cl. A61K 31/216; A61K 36/25; A61K 45/06; A61P 35/00

(52) U.S. Cl. CPC A61K 31/216; A61K 36/25; A61K 45/06; A61P 35/00; A61K 2236/333; A61K 2236/39

(57) ABSTRACT

Provided is a pharmaceutical composition for preventing or treating breast cancer including a compound derived from Dendropanax morbiferus. It was confirmed that dihydroconiferyl ferulate, a compound derived from Dendropanax morbiferus of the present disclosure, inhibited the expression of c-Myc known to be characteristically expressed in breast cancer, inhibited the mammosphere formation of breast cancer stem cells, and inhibited an EGFR signaling pathway. Accordingly, the compound inhibits the proliferation of breast cancer cells and inhibits the growth of cancer stem cells, and thus can be usefully used for prevention or treatment of breast cancer.

### CROSS-REFERENCE TO RELATED APPLICATIONS

This application is based on and claims priority from Korean Patent Application No. KR10-2022-0120436, filed on Sep. 23, 2022, with the Korean Intellectual Property Office, the disclosure of which is incorporated herein in its entirety by reference.

### REFERENCE TO AN ELECTRONIC SEQUENCE LISTING

The contents of the electronic sequence listing (Q286112 sequence listing as filed .xml; Size: 11,752 bytes; and Date of Creation: May 22, 2023) is herein incorporated by reference in its entirety.

### TECHNICAL FIELD

The present disclosure relates to a pharmaceutical composition for preventing or treating breast cancer including a compound derived from Dendropanax morbiferus.

### BACKGROUND

As chemotherapy fails to effectively target and treat cell populations within tumors, leading to recurrence and metastasis of tumors, interest in cancer stem cells has emerged. Many cytotoxic anticancer drugs usually target rapidly proliferating cells, so that cancer stem cells with slow proliferating characteristics may survive in cytotoxic anticancer therapy. Basal cell phenotype breast cancer is thought to originate from the earliest mammary progenitor cells in the early stage of a differentiation process, and is known to have a poor prognosis and show resistance to conventional chemotherapy, but this is a good example to support the fact that the cause of cancer treatment failure is the failure of targeted therapy for cancer stem cells.

Several treatment methods have been devised based on a cancer stem cell hypothesis, and among them, the most known method is a method using a self-renewal pathway of cancer stem cells. An important point in this treatment is to target only the self-renewal of cancer stem cells while maintaining the self-renewal of normal stem cells. For example, a Notch signal is processed by an enzyme called secretase, and a tumor suppression effect can be exhibited when a secretase inhibitor is used in breast cancer in which Notch1 is overexpressed. There is a recent report that even when targeting a Hedgehog signaling system, an anticancer effect is exhibited, and when cyclopamine, a Hedgehog inhibitor, was administered to a tumor xenograft animal, the tumor has dramatically atrophied.

On the other hand, breast cancer is a common cancer in women and is known to be a leading cause of death in female cancer patients (al A, Bray F, Center M M, Ferlay J, Ward E and Forman D. Globalcancer statistics. CA Cancer J Clin. 2011; 61(2):69-90). Polychemotherapy for early breast cancer, extensive mammography with tamoxifen, and adjuvant therapy have reduced the mortality rate of breast cancer, but breast cancer is still known as the most dangerous disease due to recurrence and metastasis.

Cancer stem cells (CSCs) were first identified in myelogenous leukemia and then found in various solid cancers, including breast, brain, colon, ovarian, pancreatic, and prostate cancers. The cancer stem cells are also called tumor-initiating cells and cancer stem-like cells. It has also been shown that various cancer types, including breast cancer, are derived from cancer stem cells (CSCs), a subgroup of tumors. These populations are known to induce changes in tumor volume through self-renewal and differentiation. Wnt (wingless), Shh (sonic hedgehog), Stat3, NF-κB, Wnt/β-catenin, TGF-β, and Notch signaling pathways are known to be critical for self-renewal of CSCs.

Cancer stem cells exhibit drug resistance and radiation resistance to chemotherapy and radiation therapy, and cause cancer recurrence and metastasis. Accordingly, targeted therapy for cancer stem cells is required for cancer therapy. Cancer stem cells are known to express a specific protein including Oct4, C-myc, Nanog, and aldehyde dehydrogenase-1 (ALDH). The ALDH is an enzyme that oxidizes genotoxic aldehydes, and its enzymatic activity is widely used as a cancer stem cell (CSC) marker for leukemia, and head and neck, bladder, bone, colon, liver, lung, pancreas, prostate, thyroid and cervical cancers. The ALDH is known as a therapeutic target for cancer stem cells. In addition, the ALDH is known to have excellent ability to form tumors in a breast cancer population expressing CD44⁺/CD24⁻ in clinical specimens (Al-Hajj M, Wicha M S, Benito-Hernandez A, Morrison S J and Clarke M F. Prospective identification of tumorigenic breast cancer cells. Proc Natl Acad Sci USA. 2003; 100(7):3983-3988).

A breast cancer cell line MCF-7 is known to have a partial colony of cells with stem cell-like ability to grow in an elliptical shape without apoptosis even without adherence in vitro. When a condition without a basic layer is artificially created by suspension culture, cells having stem cell properties are attached to each other to form a spherical cell mass, and such a cell mass is named a neurosphere. “Mammosphere” is an application of this concept to human breast stem cells. There are 8 times more progenitor cells than normal human breast cells in mammospheres, and continuous subculture is possible, and after several passages, 100% of the cells grow into bi-potent precursors. All of the mammospheres may differentiate into adult breast cells such as mammary gland epithelial cells, ductal epithelial cells, and alveolar epithelial cells, and it is observed that a complex functional breast structure is formed while forming a three-dimensional structure in a Matrigel. Since the mammospheres have a property of self-renewal, which is one of the most characteristic features of stem cells, multiple mammospheres or breast stem cells can be obtained in large quantities from one mammosphere. In addition, it was confirmed that many expression genes were overlapped compared to hematopoietic stem cells, neural stem cells, embryonic stem cells, etc., and it was reported that mammospheres are actual breast stem cells. Standard analysis methods for the self-renewal ability of cancer stem cells are to analyze in vivo transplantation and in vitro mammosphere formation.

Until now, research on cancer stem cells has many limitations, and nothing has been clearly identified about their role in the formation or maintenance of tumors. In order to efficiently perform treatment targeting only cancer stem cells without damaging normal stem cells, knowledge and understanding of molecular biological characteristics important for the maintenance and control of cancer stem cells or their regulatory pathways are required.

Until now, there has been little research on anticancer drugs or natural product-derived extracts that directly target cancer stem cells. In the related art, research on inhibiting cancer stem cells by an experiment suppressing direct target genes of cancer stem cells or suppressing upper signaling proteins of cancer stem cells has been conducted. However, these targeting experiments had many difficulties due to mutations of oncogenes or mutations of proteins in many tumor patients.

On the other hand, Dendropanax morbiferus H.Lev is a flowering plant belonging to the Asteraceae family and is known as a traditional medicinal plant in Korea, China and South America. The edible parts of Dendropanax morbiferus, such as leaves, bark, roots and stems, are known to prevent various diseases.

Accordingly, the present inventors isolated various compounds from Dendropanax morbiferus having various pharmacological activities and confirmed that among the compounds, dihydroconiferyl ferulate had inhibitory activity against breast cancer stem cells, and then completed the present disclosure.

### SUMMARY

The present disclosure has been made in an effort to provide a pharmaceutical composition for preventing or treating breast cancer including a Dendropanax morbiferus extract or a fraction thereof as an active ingredient.

The present disclosure has also been made in an effort to provide a pharmaceutical composition for preventing or treating breast cancer including dihydroconiferyl ferulate or a pharmaceutically acceptable salt thereof as an active ingredient.

The present disclosure has also been made in an effort to provide an anticancer adjuvant for enhancing the sensitivity to an anticancer agent including dihydroconiferyl ferulate or a pharmaceutically acceptable salt thereof as an active ingredient

The present disclosure has also been made in an effort to provide a food composition for preventing or improving breast cancer including dihydroconiferyl ferulate or a food acceptable salt thereof as an active ingredient.

An embodiment of the present disclosure provides a pharmaceutical composition for preventing or treating breast cancer including a Dendropanax morbiferus extract or a fraction thereof as an active ingredient.

Another embodiment of the present disclosure provides a pharmaceutical composition for preventing or treating breast cancer including dihydroconiferyl ferulate or a pharmaceutically acceptable salt thereof as an active ingredient

Yet another embodiment of the present disclosure provides an anticancer adjuvant for enhancing the sensitivity to an anticancer agent including dihydroconiferyl ferulate or a pharmaceutically acceptable salt thereof as an active ingredient

Still another embodiment of the present disclosure provides a food composition for preventing or improving breast cancer including dihydroconiferyl ferulate or a food acceptable salt thereof as an active ingredient.

According to the embodiments of the present disclosure, dihydroconiferyl ferulate inhibited the formation of breast cancer stem cells. In addition, it was confirmed that the expression of c-Myc, which was known to be characteristically expressed in breast cancer, was inhibited, and mammosphere formation of breast cancer stem cells was inhibited, and an EGFR signaling pathway was inhibited. Accordingly, the compound inhibits the proliferation of breast cancer cells and inhibits the growth of cancer stem cells, and thus can be usefully used for prevention or treatment of breast cancer.

The foregoing summary is illustrative only and is not intended to be in any way limiting. In addition to the illustrative aspects, embodiments, and features described above, further aspects, embodiments, and features will become apparent by reference to the drawings and the following detailed description.

### BRIEF DESCRIPTION OF THE DRAWINGS

FIG. 1 is a schematic diagram briefly showing a process of isolating a Dendropanax morbiferus-derived breast cancer stem cell inhibitor according to an embodiment of the present disclosure and a diagram illustrating a result of mammosphere formation analysis and an HPLC analysis result of a purified sample.

FIG. 2 is a diagram illustrating results of silica gel column chromatography of ethyl acetate fractions for a Dendropanax morbiferus methanol extract according to an embodiment of the present disclosure.

FIG. 3 is a diagram illustrating results of LH-20 gel column chromatography of Fraction #4 obtained by silica gel column chromatography according to an embodiment of the present disclosure.

FIG. 4 is a diagram illustrating results of thin layer chromatography (TLC) and UV irradiation for four fractions obtained by LH-20 gel column chromatography according to an embodiment of the present disclosure.

FIG. 5 is a diagram illustrating an NMR analysis result for a dihydroconiferyl ferulate compound isolated and purified from Dendropanax morbiferus according to an embodiment of the present disclosure.

FIG. 6 is a diagram illustrating results of analyzing breast cancer cell proliferation inhibitory ability, mammosphere formation inhibitory ability, and cell migration inhibitory ability of dihydroconiferyl ferulate according to an embodiment of the present disclosure.

FIG. 7 is a diagram illustrating results of a CD44⁺/CD24⁻ flow cytometry assay, an apoptosis assay, and qRT-PCR for cancer stem cell-specific markers and a mammosphere formation assay in breast cancer cells treated with dihydroconiferyl ferulate according to an embodiment of the present disclosure.

FIG. 8 is a diagram illustrating results of evaluating total protein and nuclear protein levels of EGFR in breast cancer cells after 48 hours of dihydroconiferyl ferulate treatment according to an embodiment of the present disclosure.

FIG. 9 is a diagram illustrating results of evaluating total protein, cytosolic protein and nuclear protein levels of p-Stat3 and Stat3 in breast cancer cells after 48 hours of dihydroconiferyl ferulate treatment according to an embodiment of the present disclosure.

FIG. 10 is a diagram illustrating results of analyzing mRNA and total protein, cytosolic protein and nuclear protein levels of c-Myc and Stat3 in breast cancer cells treated with dihydroconiferyl ferulate treatment according to an embodiment of the present disclosure.

FIG. 11 is a schematic diagram briefly illustrating a mechanism in which dihydroconiferyl ferulate inhibits the formation of breast cancer stem cells through an EGFR-Stat3/c-Myc signaling pathway according to an embodiment of the present disclosure.

### DETAILED DESCRIPTION

In the following detailed description, reference is made to the accompanying drawing, which forms a part hereof. The illustrative embodiments described in the detailed description, drawing, and claims are not meant to be limiting. Other embodiments may be utilized, and other changes may be made, without departing from the spirit or scope of the subject matter presented here.

Hereinafter, an embodiment of the present disclosure will be described in detail with reference to the accompanying drawings. However, the following exemplary embodiments are presented as examples for the present disclosure, and when it is determined that a detailed description of well-known technologies or configurations known to those skilled in the art may unnecessarily obscure the gist of the present disclosure, the detailed description thereof may be omitted, and the present disclosure is not limited thereto. Various modifications and applications of the present disclosure are possible within the description of claims to be described below and the equivalent scope interpreted therefrom.

Terminologies used herein are terminologies used to properly express embodiments of the present disclosure, which may vary according to a user, an operator's intention, or customs in the art to which the present disclosure pertains. Accordingly, definitions of the terminologies need to be described based on contents throughout this specification. Throughout the specification, when a part “comprises” a certain component, it is meant that the part may further include other components, not excluding other components, unless explicitly described to the contrary.

Throughout this specification, ‘%’ used to indicate the concentration of a specific material is solid/solid (w/w) %, solid/liquid (w/v) %, and liquid/liquid (v/v) %, unless otherwise stated.

Hereinafter, the present disclosure will be described in detail.

The present disclosure provides a pharmaceutical composition for preventing or treating breast cancer including a Dendropanax morbiferus extract or a fraction thereof as an active ingredient.

In an embodiment, Dendropanax morbiferus may be Jeju Dendropanax morbiferus obtained from the Jeju Special Self-Governing Province, Korea, but is not limited thereto.

In an embodiment, the extract may be extracted from the roots, stems or leaves of Dendropanax morbiferus, preferably from the leaves of Dendropanax morbiferus, but is not limited thereto.

The term “extract” used in the present disclosure refers to a preparation obtained by squeezing herb medicine into an appropriate leachate and evaporating and concentrating the leachate, and is meant to be commonly used as a crude extract in the art, but includes widely fractions fractionating further the extract. That is, the Dendropanax morbiferus extract includes not only those obtained using the above-described extraction solvent, but also those obtained by further applying a purification process thereto. For example, the extract also includes a fraction obtained by passing the extract through an ultrafiltration membrane having a certain molecular weight cut-off value, and fractions obtained by various purification methods to be further performed, such as separation by various chromatography (made for separation according to size, charge, hydrophobicity or affinity), etc. In addition, the extract is not limited thereto, but may be an extract obtained by extraction treatment, a diluted or concentrated extract of the extract, a dried product obtained by drying the extract, and a crude or purified product thereof. The Dendropanax morbiferus extract may be prepared using general extraction methods, and separation and purification methods known in the art. The extraction methods are not limited thereto, but may preferably use methods, such as boiling water extraction, hot water extraction, chilling extraction, reflux cooling extraction, or ultrasonic extraction.

In an embodiment, the extract may be extracted with one or more solvents selected from the group consisting of water, organic solvents, subcritical fluids and supercritical fluids, and may be extracts extracted with at least one extraction solvent selected from the group consisting of water, anhydrous or hydrous alcohol having 1 to 4 carbon atoms, ethyl acetate, acetone, glycerin, ethylene glycol, propylene glycol and butylene glycol. The organic solvent may be at least one organic solvent selected from the group consisting of lower alcohol having 1 to 4 carbon atoms, hexane (n-hexane), ether, glycerol, propylene glycol, butylene glycol, ethyl acetate, methyl acetate, dichloromethane, chloroform, ethyl acetate, acetone, methylene chloride, cyclohexane, petroleum ether and benzene, preferably methanol, but is not limited thereto.

In addition, the Dendropanax morbiferus extract obtained above is suspended in water, and then systematically isolated using an extraction solvent according to a conventional method, and concentrated under reduced pressure to obtain fractions of the Dendropanax morbiferus extract for each extraction solvent.

In an embodiment, the fraction may be an ethanol fraction, a methanol fraction, a dichloromethane fraction, an ethyl acetate fraction, a water fraction, an n-hexane fraction, a chloroform fraction, an ethyl acetate fraction, or a butanol fraction, but is not limited thereto.

In an embodiment, the composition may inhibit the growth of breast cancer stem cells, inhibit the formation of breast cancer-derived mammospheres, or suppress the proliferation of breast cancer-derived mammospheres, and inhibit the expression of a c-Myc gene or protein, but is not limited thereto.

In an embodiment, the breast cancer may be breast cancer expressing CD44^{high}/CD24^{low}, but is not limited thereto.

The composition of the present disclosure further includes not only a Dendropanax morbiferus extract or a fraction thereof, but also other active ingredients having the same or similar functions, or other active ingredients having functions different from those of the ingredients to be prepared as a pharmaceutical composition for preventing or treating breast cancer.

The “prevention” means any action that reduces the frequency or severity of pathological phenomena. The prevention may be complete or partial. In this case, the prevention may mean a phenomenon in which the symptoms of breast cancer in the subject are reduced compared to the case where the composition is not used.

The “treatment” means any action that clinically intervenes to change a natural process of a target or cell to be treated, and may be performed while a clinical pathological condition is progressing or to prevent it. The desired therapeutic effect may include preventing the occurrence or recurrence of the disease, alleviating the symptoms, reducing any direct or indirect pathological consequences according to a disease, preventing metastasis, reducing a disease progression rate, reducing or temporarily alleviating a disease condition, or improving the prognosis.

The pharmaceutical composition of the present disclosure is administered in a pharmaceutically effective dose. The term ‘pharmaceutically effective dose’ used herein refers to an amount enough to treat the disease at a reasonable benefit/risk ratio applicable to medical treatment. An effective dose level may be determined according to factors including the health condition of a patient, the type and severity of a disease, the activity of a drug, the sensitivity to a drug, a method of administration, a time of administration, a route of administration, an emission rate, duration of treatment, and combined or simultaneously used drugs, and other factors well-known in the medical field. The composition of the present disclosure may be administered as an individual therapeutic agent or in combination with other therapeutic agents, and may be administered sequentially or simultaneously with existing therapeutic agents, and may be administered singly or multiply. It is important to administer an amount capable of obtaining a maximum effect with a minimal amount without side-effects by considering all the factors, which may be easily determined by those skilled in the art.

In the present disclosure, the “subject” is not particularly limited as long as the subject is any object for the purpose of preventing or treating breast cancer, and includes mammal animals including humans, such as non-primates (e.g., cow, pig, horse, cat, dog, rats and mice) and primates (e.g., monkeys such as cynomolgus monkeys and chimpanzees). In some cases, the subject may be a subject excluding humans.

The composition according to the present disclosure may include a pharmaceutically effective dose as an active ingredient alone or may include one or more pharmaceutically acceptable carriers, excipients or diluents. The pharmaceutically effective dose refers to an amount sufficient to prevent, improve, and treat symptoms of breast cancer. The “pharmaceutically acceptable” refers to a composition that is physiologically acceptable and does not cause an allergic reaction, such as gastrointestinal disorder, dizziness, etc., or a similar reaction thereto when administered to humans.

The composition including the pharmaceutically acceptable carrier may have various oral or parenteral formulations. When the composition is formulated, the formulations may be prepared by using diluents or excipients, such as a filler, an extender, a binder, a wetting agent, a disintegrating agent, a surfactant, etc., which are generally used. The carriers, excipients and diluents may be at least one selected from the group consisting of lactose, dextrose, sucrose, sorbitol, mannitol, xylitol, erythritol, maltitol, starch, gum acacia, alginate, gelatin, calcium phosphate, calcium silicate, cellulose, methyl cellulose, microcrystalline cellulose, polyvinyl pyrrolidone, physiological saline, methylhydroxybenzoate, propylhydroxybenzoate, talc, magnesium stearate and mineral oil, dextrin, calcium carbonate, propylene glycol, and liquid paraffin, but are not limited thereto, and may be used as all conventional carriers, excipients or diluents. The components may be added independently or in combination with the active ingredient, which is the active ingredient.

Solid formulations for oral administration may include tablets, pills, powders, granules, capsules, and the like, and the solid formulations may be prepared by mixing at least one excipient, for example, starch, calcium carbonate, sucrose or lactose, gelatin, and the like with at least one compound. Further, lubricants such as magnesium stearate and talc may also be used in addition to simple excipients. Liquid formulations for oral administration may correspond to a suspension, an oral liquid, an emulsion, a syrup, and the like, and may include various excipients, for example, a wetting agent, a sweetener, an aromatic agent, a preserving agent, and the like, in addition to water and liquid paraffin which are commonly used as simple diluents.

Formulations for parenteral administration include a sterile aqueous solution, a non-aqueous solution, a suspension, an emulsion, a lyophilizing agent, a suppository, and the like. As the non-aqueous solution and the suspension, propylene glycol, polyethylene glycol, vegetable oil such as olive oil, injectable ester such as ethyl oleate, and the like may be used. As a base of the suppository, witepsol, macrogol, Tween 61, cacao butter, laurinum, glycerol, gelatin, and the like may be used.

Further, the pharmaceutical composition of the present disclosure may have any one formulation selected from the group consisting of tablets, pills, powders, granules, capsules, suspensions, oral liquids, emulsions, syrups, sterilized aqueous solutions, non-aqueous solvents, lyophilized agents, and suppositories. As a base of the suppository, witepsol, macrogol, Tween 61, cacao butter, laurinum, glycerol, gelatin, and the like may be used.

The active ingredients of the present disclosure may be administered in various oral and parenteral formulations during clinical administration, and for formulations, may be prepared by using commonly used diluents or excipients, such as a filler, an extender, a binder, a wetting agent, a disintegrant, and a surfactant.

Solid formulations for oral administration include a tablet, a pill, a powder, a granule, a capsule, troche, and the like, and the solid formulations may be prepared by mixing at least one excipient, for example, starch, calcium carbonate, sucrose, lactose, gelatin, or the like with one or more active ingredients of the present disclosure. Further, lubricants such as magnesium stearate and talc may be used in addition to simple excipients. Liquid formulations for oral administration may correspond to suspensions, oral liquids, emulsions, syrups, and the like, and may include various excipients, for example, a wetting agent, a sweetener, an aromatic agent, a preserving agent, and the like, in addition to water and liquid paraffin which are commonly used simple diluents.

Formulations for parenteral administration include a sterile aqueous solution, a non-aqueous solution, a suspension, an emulsion, a lyophilizing agent, a suppository, and the like. As the non-aqueous solution and the suspension, propylene glycol, polyethylene glycol, vegetable oil such as olive oil, injectable ester such as ethyl oleate, and the like may be used. As a base of the suppository, witepsol, macrogol, Tween 61, cacao butter, laurinum, glycerol, gelatin, and the like may be used.

In addition, the effective dose for the human body of the active ingredient of the present disclosure may vary depending on the age, weight, sex, dosage form, health condition and disease degree of a patient, and may be generally about 0.001 to 100 mg/kg/day, preferably 0.01 to 35 mg/kg/day. Based on an adult patient with a body weight of 70 kg, the effective dose may be generally 0.07 to 7000 mg/day, preferably 0.7 to 2500 mg/day, and may be divided once or several doses a day at regular intervals according to the judgment of a doctor or pharmacist.

The pharmaceutical composition of the present disclosure may be used alone or in combination with methods using surgery, radiation therapy, hormone therapy, chemotherapy or biological response modifiers.

The pharmaceutical composition of the present disclosure may also be provided in the form of an external preparation including a Dendropanax morbiferus extract or a fraction thereof as an active ingredient.

When the pharmaceutical composition of the present disclosure is used as an external skin preparation, the pharmaceutical composition may further include adjuvants commonly used in the field of dermatology, such as any other ingredients commonly used in external skin preparations, such as fatty substances, organic solvents, solubilizers, thickening and gelling agents, emollients, antioxidants, suspending agents, stabilizers, foaming agents, fragrances, surfactants, water, ionic emulsifiers, nonionic emulsifiers, fillers, sequestering agents, chelating agents, preservatives, vitamins, blockers, wetting agents, essential oils, dyes, pigments, hydrophilic activators, lipophilic activators or lipid vesicles. In addition, the ingredients may be introduced in an amount generally used in the field of dermatology. When the pharmaceutical composition of the present disclosure is provided as an external skin preparation, the external skin preparation is not limited thereto, but may be a formulation such as ointments, patches, gels, creams, or sprays.

Next, the present disclosure provides a pharmaceutical composition for preventing or treating breast cancer including dihydroconiferyl ferulate or a pharmaceutically acceptable salt thereof as an active ingredient.

In an embodiment, the dihydroconiferyl ferulate may be represented by Chemical Formula 1 below, but is not limited thereto.

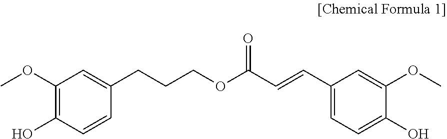

In an embodiment, the composition may inhibit the growth of breast cancer stem cells. Accordingly, the dihydroconiferyl ferulate of the present disclosure may be used as a composition for inhibiting the growth of breast cancer stem cells.

In the present disclosure, the “breast cancer stem cells” means cancer stem cells present in breast cancer tissue. The “cancer stem cell” is an undifferentiated cell having the ability to differentiate into various cancer cells, and the cancer may be colorectal cancer including colon cancer and rectal cancer, breast cancer, uterine cancer, cervical cancer, ovarian cancer, prostate cancer, brain tumor, head and neck carcinoma, melanoma, myeloma, leukemia, lymphoma, stomach cancer, lung cancer, pancreatic cancer, liver cancer, esophageal cancer, small intestine cancer, perianal cancer, fallopian tube carcinoma, endometrial carcinoma, vaginal carcinoma, vulvar carcinoma, Hodgkin's disease, bladder cancer, kidney cancer, ureteral cancer, renal cell carcinoma, renal pelvic carcinoma, bone cancer, skin cancer, head cancer, cervical cancer, skin melanoma, intraocular melanoma, endocrine cancer, thyroid cancer, parathyroid cancer, adrenal cancer, soft tissue sarcoma, urethral cancer, penile cancer, central nervous system (CNS) tumor, primary CNS lymphoma, spinal cord tumor, brainstem glioma or pituitary adenoma. More preferably, the cancer may be breast cancer.

In the present disclosure, the “inhibition of the cancer stem cell growth” includes inhibition of cancer stem cell maintenance, inhibition of cancer stem cell malignance, and inhibition of cancer stem cell migration and cancer stem cell invasion.

In addition, the “prevention of the cancer stem cells” may refer to any action that prevents or delays the growth or proliferation of cancer stem cells by administering the composition according to the present disclosure to a subject.

In an embodiment, the prevention of the cancer stem cells may be inhibiting the formation of breast cancer-derived mammospheres, or inhibiting the proliferation of breast cancer-derived mammospheres, and inhibiting the expression of a c-Myc gene or protein, but is not limited thereto.

In an embodiment, the breast cancer may be breast cancer expressing CD44^{high}/CD24^{low}, but is not limited thereto.

In an embodiment, the composition of the present disclosure may include dihydroconiferyl ferulate at a concentration of 0.01 to 1,000 μM.

The dihydroconiferyl ferulate of the present disclosure may be used in the form of a pharmaceutically acceptable salt.

As used herein, the term “pharmaceutically acceptable salt” refers to any salt that retains the desired biological and/or physiological activity of the compound and exhibits an undesirable toxicological effect to a minimum. The pharmaceutically acceptable salt means a salt prepared according to a conventional method in the art, and such a preparation method is known to those skilled in the art.

As the salt, acid addition salts formed with pharmaceutically acceptable free acids are useful. The expression of the pharmaceutically acceptable salt is a concentration that has a relatively non-toxic and harmless effective effect on patients, and means any organic or inorganic addition salt of a base compound of dihydroconiferyl ferulate which does not deteriorate the beneficial effects of the base compound of dihydroconiferyl ferulate by adverse effects resulting from this salt. These salts may use inorganic acids and organic acids as free acids, the inorganic acid include hydrochloric acid, hydrobromic acid, nitric acid, sulfuric acid, perchloric acid, and phosphoric acid, and the organic acid may include citric acid, acetic acid, lactic acid, maleic acid, fumaric acid, gluconic acid, methanesulfonic acid, glycolic acid, succinic acid, tartaric acid, galacturonic acid, embonic acid, glutamic acid, aspartic acid, oxalic acid, (D) or (L) malic acid, maleic acid, methanesulfonic acid, ethanesulfonic acid, 4-toluenesulfonic acid, salicylic acid, citric acid, benzoic acid or malonic acid and the like. In addition, these salts include alkali metal salts (sodium salt, potassium salt, etc.), alkaline earth metal salts (calcium salt, magnesium salt, etc.), and the like. For example, the acid addition salts may include acetate, aspartate, benzate, besylate, bicarbonate/carbonate, bisulfate/sulfate, borate, camsylate, citrate, edisylate, esylate, formate, fumarate, gluceptate, gluconate, glucuronate, hexafluorophosphate, hybenzate, hydrochloride/chloride, hydrobromide/bromide, hydroiodide/iodide, isethionate, lactate, malate, maleate, malonate, mesylate, methyl sulfate, naphthylate, 2-naphsylate, nicotinate, nitrate, orotate, oxalate, palmitate, pamoate, phosphate/hydrogen phosphate/dihydrogen phosphate, saccharate, stearate, succinate, tartrate, tosylate, trifluoroacetate, aluminum, arginine, benzathine, calcium, choline, diethylamine, diolamine, glycine, lysine, magnesium, meglumine, olamine, potassium, sodium, tromethamine, zinc salts, etc., and preferably hydrochloride or trifluoroacetate among them.

The acid addition salt according to the present disclosure may be prepared by a conventional method, for example, a method of dissolving dihydroconiferyl ferulate in an organic solvent such as methanol, ethanol, acetone, methylene chloride, acetonitrile, etc. and filtering and drying a precipitate produced by adding an organic or inorganic acid, or distilling the solvent and excess acid under reduced pressure, and then drying or crystallizing the mixture under an organic solvent.

In addition, the bases may also be used to prepare pharmaceutically acceptable metal salts. An alkali metal salt or an alkaline earth metal salt may be obtained, for example, by dissolving the compound in an excess alkali metal hydroxide or alkaline earth metal hydroxide solution, filtering a non-dissolved compound salt, and then evaporating and drying a filtrate. At this time, as the metal salt, it is pharmaceutically suitable to prepare a sodium, potassium or calcium salt. Further, silver salts corresponding thereto may be obtained by reacting the alkali metal or alkaline earth metal salt with a suitable silver salt (e.g., silver nitrate).

Furthermore, the present disclosure includes not only dihydroconiferyl ferulate and a pharmaceutically acceptable salt thereof, but also all solvates, hydrates, isomers, optical isomers and the like that can be prepared therefrom.

Furthermore, the present disclosure provides an anticancer adjuvant for enhancing the sensitivity to an anticancer agent including dihydroconiferyl ferulate or a pharmaceutically acceptable salt thereof as an active ingredient.

Since the anticancer adjuvant of the present disclosure includes the above-described dihydroconiferyl ferulate, the duplicated contents with the above-described dihydroconiferyl ferulate of the present disclosure will be omitted in order to avoid excessive complexity of this specification by the duplicated contents.

In an embodiment, the anticancer agent may be a chemotherapeutic drug and may be an anti-cell division drug.

In an embodiment, the anticancer agent may be eribulin, carboplatin, cisplatin, Halaven, 5-fluorouracil (5-FU), gleevec, Vincristine, Vinblastine, Vinorelvine, Paclitaxel, Docetaxel, Etoposide, Topotecan, Irinotecan, Dactinomycin, Doxorubicin, Daunorubicin, valrubicin, flutamide, gemcitabine, Mitomycin, or Bleomycin, but is not limited thereto.

In an embodiment, the cancer may be cancer stem cell, multi-drug resistant cancer or anticancer drug resistant cancer.

In an embodiment, the cancer may be a resistant cancer by formation of cancer stem cells.

In an embodiment, the anticancer adjuvant of the present disclosure may increase apoptosis of cancer cells, multi-drug resistant cancer cells or cancer stem cells.

In an embodiment, the anticancer adjuvant of the present disclosure may be administered in combination with an anticancer agent, and may be administered simultaneously with, separately from, or sequentially with the anticancer agent.

In an embodiment, the cancer may be any one or more selected from the group consisting of brain tumor, melanoma, myeloma, non-small cell lung cancer, oral cancer, liver cancer, stomach cancer, colon cancer, breast cancer, lung cancer, bone cancer, pancreatic cancer, skin cancer, head or neck cancer, cervical cancer, ovarian cancer, colorectal cancer, small intestine cancer, rectal cancer, fallopian tube carcinoma, perianal cancer, endometrial carcinoma, vaginal carcinoma, vulvar carcinoma, Hodgkin's disease, esophageal cancer, lymphoma, bladder cancer, gallbladder cancer, endocrine cancer, thyroid cancer, parathyroid cancer, adrenal cancer, soft tissue sarcoma, urethral cancer, penile cancer, prostate cancer, chronic or acute leukemia, lymphocytic lymphoma, kidney or ureter cancer, renal cell carcinoma, renal pelvic carcinoma, central nervous system tumor, primary central nervous system lymphoma, spinal cord tumor, brainstem gliomas and pituitary adenomas, preferably breast cancer.

The term “anticancer adjuvant” used herein is an agent that may improve, enhance or increase an anticancer effect of an anticancer agent, and does not show anticancer activity by itself, but when used together with an anticancer agent, it may be an agent capable of improving, enhancing or increasing the anticancer effect of the anticancer agent. In addition, when an agent exhibiting a concentration-dependent anticancer activity is used together with an anticancer agent at a level that does not exhibit anticancer activity by itself, it may be an agent that can improve, enhance or increase the anticancer effect of the anticancer agent.

The route of administration of the anticancer adjuvant may be administered through any general route as long as the anticancer adjuvant may reach a target tissue. The anticancer adjuvant of the present disclosure may be administered intraperitoneally, intravenously, intramuscularly, subcutaneously, intradermally, orally, intranasally, pulmonary, or intrarectally according to a desired purpose, but is not limited thereto. In addition, the anticancer adjuvant may be administered by any device capable of transferring an active ingredient to a target cell.

Further, the present disclosure provides a food composition for preventing or improving breast cancer including dihydroconiferyl ferulate or a food acceptable salt thereof as an active ingredient.

The food composition of the present disclosure may be formulated into various forms such as tablets, pills, granules, capsules, liquid preparations, and beverages and added to foods. The kind of food is not particularly limited. Examples of the food which may be added with the dihydroconiferyl ferulate of the present disclosure include drinks, meat, sausages, bread, biscuits, rice cakes, chocolate, candies, snacks, confectionery, pizza, ramen, other noodles, gums, dairy products including ice cream, various soups, beverages, alcohol beverages and vitamin complex, dairy products and processed dairy products, and the like, and include both health foods and health functional foods in an accepted meaning.

The composition in the health food and health functional food containing the dihydroconiferyl ferulate according to the present disclosure may be added to the foods as it is or used with other foods or food ingredients, and may be appropriately used according to a general method. The mixing amount of the dihydroconiferyl ferulate may be suitably determined according to the purpose of use thereof (prevention or improvement). In general, the composition in the health food and the health functional food may be added in an amount of 0.1 to 90 parts by weight of the total food weight. However, in the case of long-term intake for the purpose of health maintenance or health control, the amount may be less than or equal to the above range, and there is no problem in terms of safety, so that the active ingredient may be used even in an amount above the range.

The composition in the health food and health functional food of the present disclosure is not particularly limited to other ingredients other than the dihydroconiferyl ferulate as an essential ingredient in an indicated ratio, and may contain various flavoring agents or natural carbohydrates as an additional ingredient, like conventional beverages. Examples of the above-mentioned natural carbohydrates may include general sugars, such as monosaccharides, for example, glucose, fructose and the like; disaccharides, for example, maltose, sucrose and the like; and polysaccharides, for example, dextrin, cyclodextrin and the like, and sugar alcohols such as xylitol, sorbitol, erythritol, and the like. As flavoring agents other than those described above, natural flavoring agents (tauumatin, stevia extract (e.g., rebaudioside A, glycyrginine, etc.)), and synthetic flavoring agents (saccharin, aspartame, etc.) may be advantageously used. A ratio of the natural carbohydrates may be generally about 1 to 20 g, preferably about 5 to 12 g per 100 of the health functional food composition of the present disclosure.

In addition, the composition in the health food and the health functional food containing the dihydroconiferyl ferulate of the present disclosure may contain various nutrients, vitamins, minerals (electrolytes), flavoring agents such as synthetic and natural flavoring agents, coloring agents and enhancers (cheese, chocolate, etc.), pectic acid and salts thereof, alginic acid and salts thereof, organic acid, a protective colloidal thickener, a pH adjusting agent, a stabilizer, a preservative, glycerin, alcohol, a carbonic acid agent used in a carbonated drink, and the like. In addition, the composition in the health food and the health functional food of the present disclosure may contain pulps for preparing natural fruit juice, fruit juice beverages, and vegetable beverages.

These ingredients may be used independently or in combination. The ratio of these additives are not so important, but may be generally selected in the range of 0.1 to about 20 parts by weight per 100 parts by weight of the composition in the health food and the health functional food containing the dihydroconiferyl ferulate of the present disclosure.

Hereinafter, Examples of the present disclosure will be described in more detail with reference to the accompanying drawings. However, the following Examples are only intended to embody the contents of the present disclosure, and the present disclosure is not limited thereto.

### <Experimental Example 1> Chemicals and Reagents

Silica gel 60 powder, silica gel 60 F₂₅₄ aluminum sheets and glass plates for thin-layer chromatography (TLC) were purchased from Merck Supelco (Darmstadt, Hesse, Germany). Sephadex LH-20 (LH20_100) power was purchased from Millipore (Cytiva, Marlborough, MA, USA), and high-pressure liquid chromatography (HPLC) was conducted using a Shimadzu LC-10 system (Tokyo, Japan). The cell viability of breast cancer cells was determined using a cell viability assay kit (EZ-Cytox, DoGenBio, Seoul, Korea). All other chemicals and organic solvents not mentioned above were purchased from Sigma (St. Louis, MO, USA) and used.

### <Experimental Example 2> Culture of Human Breast Cancer Cells and Mammospheres

Human breast cancer cell lines, MDA-MB-231 and MCF-7 cells, were purchased from the Korea Cell Line Bank and cultured in an incubator with an atmospheric environment of 5% CO₂ and a Dulbecco's Modified Eagle's Medium (DMEM) containing 10% fetal bovine serum (Corning, Glendale, AZ, USA) and 1% penicillin/streptomycin.

MDA-MA-231 cells (1×10⁴ per well) and MCF-7 cells (4×10⁴ per well) were incubated in a 6-well ultralow attachment plate with a MammoCult™ culture medium (StemCell Technologies, Vancouver, BC, Canada) containing hydrocortisone (0.5 μg/mL) and heparin (4 μg/mL) for 7 days.

### <Experimental Example 3> Cell Proliferation and Mammosphere Formation Assay

MCF-7 cells (1.5×10⁶ cells/plate) and MDA-MB-231 cells (1.0×10⁶ cells/plate) were seeded into a 96-well plate for 24 hours and incubated with dihydroconiferyl ferulate (0, 10, 25, 50, 75, 100, 150, and 200 μM). Thereafter, cell viability was examined using the EZ-Cytox Plus Kit (DoGenBio, Seoul, Korea) according to the manufacturer's protocol. OD₄₅₀ values were measured using a VERSAₘₐₓ ELISA reader (Molecular Device, San Jose, CA, USA).

The mammosphere formation was quantified using an integrated colony enumerator (NICE) program of NIST, and the rate of mammosphere formation was measured by mammosphere formation efficiency (MFE). Here, the MFE (%) was calculated by the following Equation.

MFE (%)=[number of spheres in control group or drug-treated group/number of spheres in control group (DMSO-treated group)]×100

### <Experimental Example 4> TLC Plate Analysis

In order to separate active components from fractions eluted by column chromatography, the concentrated fractions were loaded onto prep TLC (glass plate; 20×20, layer thickness; 210 to 270 um) and developed on a mixed solvent (30:1) of chloroform and methanol for 2 hours in a TLC glass chamber. Then, after the plate was dried, UV radiation (UV254 nm and UV365 nm) was irradiated to visualize fluorescence. Each band was separated from a silica gel plate, and ingredients attached to the silica gel were dissolved using methanol, and a methanol eluate was concentrated by filtration. Each fraction was analyzed for mammosphere formation.

### <Experimental Example 5> Cell Colony Formation and Cell Migration Assay

5-1. Cell Colony Formation Method

MDA-MB-231 cells (5×10² cells/well) were cultured in a 6-well plate for 7 days with a caudatin compound at different concentrations (50, 100, 150, 300 and 400 uM). Thereafter, colonies were then immobilized with 3.7% formaldehyde for 10 minutes and stained with 0.05% crystal violet for 30 minutes. The grown colonies were analyzed using a scanner (Umax PowerLook 1100, lasersoft Imaging, Seoul, Korea).

5-2. Cell Migration Assay

MDA-MB-231 cancer cells were dispensed in a 6-well plate at a cell count of 2×10⁶ with DMEM/10% FBS. When the cells grew as a monolayer, the cells were scratched using a microtip. After washed twice with 1×PBS, the cells were added with 50 or 100 uM of caudatin together with a fresh DMEM/0.5% FBS medium and cultured for 12 hours. Micrographs of the wound site were analyzed with an optical microscope. A white line on which the cells migrated across was measured for 5 randomly selected areas, and an inhibition rate (%) was calculated by taking untreated wells as 100%.

### <Experimental Example 6> CD44⁺/CD24⁻ Flow Cytometry

MDA-MB-231 cancer cells (1.5×10 6 cells) were cultured in a 6-well plate for 24 hours and treated with DMSO as a control group or dihydroconiferyl ferulate (50 μM) for 24 hours. Thereafter, the cells isolated into single cells were incubated with an anti-human CD44 antibody (FITC-conjugated) and an anti-human CD24 antibody (PE-conjugated) (BD) at 4° C. for 45 minutes. After washing with 1×PBS, CD44⁺/CD24⁻ cells were examined using a flow cytometer Accuri C6 machine (BD San Jose, CA, USA).

### <Experimental Example 7> Apoptosis Assay

Mammospheres formed from MDA-MB-231 cells were treated with dihydroconiferyl ferulate (50 μM) for 24 hours. Apoptosis was measured in mammospheres treated with dihydroconiferyl ferulate using an Annexin V Apoptosis Detection kit containing PI(BD) according to a manufacturer's protocol. Mammospheres were collected and separated with 0.05% Trypsin-EDTA IX (Corning).

Briefly, 1×10⁶ cells were put in a binding buffer containing Annexin V (FITC) and PI, incubated at room temperature for 30 minutes by blocking light, and then examined by flow cytometry at the Jeju Center of Korea Basic Science Institute. Mammospheres derived from MDA-MB-231 cells cultured with or without dihydroconiferyl ferulate (50 μM) were isolated into single cells, and an equal number of cancer cells were seeded in a 6-well plate. The number of cells was counted at 24 hours, 48 hours, and 72 hours to measure the growth of mammospheres.

### <Experimental Example 8> Western Blot Analysis

Protein samples of mammospheres formed in MDA-MB-231 cells (1×10 4 per well) treated with dihydroconiferyl ferulate (501.1M) were obtained, and subjected to 10% SDS gel electrophoresis (SDS-PAGE), and then electrotransferred to polyvinylidene difluoride membranes (Millipore, Billerica, MA, USA). Then, the membrane was incubated in a blocking buffer containing a primary antibody overnight at 4° C. The primary antibody used anti-EGFR (#4267s, Cell Signaling Technology, Denver, CO, USA), anti-pStat3 (#9145s, Cell Signaling Technology, Denver, CO, USA), anti-c-Myc (#5605s, Cell Signaling Technology, Denver, CO, USA), anti-Stat3 (sc-482), anti-Lamin B (sc-6216) and anti-β-actin (sc-47778 Santa Cruz Biotechnology, Dallas, TX, USA). The membrane was washed with PBST (phosphate-buffered saline with Tween 20, 0.1%, v/v), and it was incubated with secondary antibodies (anti-rabbit (IRDye 800CW-conjugated), and anti-goat (IRDye 680RD-conjugated) or anti-mouse (IRDye 680RD-conjugated)). Signal images were analyzed with Odyssey CLx (Li-Cor, Lincoln, NE, USA), and concentration measurements of western blot were analyzed using an Image Studio Ver 5.2 program (Li-Cor, Lincoln, NE, USA) of Odyssey CLx.

### <Experimental Example 9> Immunoprecipitation (IP)

Protein samples of mammospheres treated with DMSO (control) or dihydroconiferyl ferulate (501.1M) were prepared in a lysis buffer (20 mM Hepes, 10 mM EGTA, 40 mM glycerol 2-phosphate, 2.5 mM MgCl₂ 6H₂O, 1% NP-40, pH 7.5) and immunoprecipitation (IP) was performed using 1 μg of a Stat3 antibody (sc-482) and 500 lag of a protein sample. Protein A/G-Agarose (P9203-100, GenDEPOT) was used to precipitate the protein complex, and analyzed using SDS-PAGE, and then immunoblotting was performed with an EGFR antibody (#4267s).

### <Experimental Example 10> RNA Extraction and qRT-PCR

Total RNA was isolated from MDA-MB-231 cells or mammospheres using a TaKaRa MiniBEST Universal RNA Extraction Kit according to the manufacturer's protocol. TOPreal™ One-step RT-qPCR kit (SYBR Green with low ROX, Enzynomics, Daejeon, Korea) was used to perform qRT-PCR, and the primers used for qRT-PCR were shown in Table 1.

TABLE 1

| Gene | Primer | |
|---|---|---|
| Nanog | Foward | 5′-ATGCCTCACACGGAGACTGT-3′ |
| | | (SEQ ID NO: 1) |
| | Reverse | 5′-AAGTGGGTTGTTTGCCTTTG-3′ |
| | | (SEQ ID NO: 2) |
| CD44 | Foward | 5′-AGAAGGTGTGGGCAGAAGAA-3′ |
| | | (SEQ ID NO: 3) |
| | Reverse | 5′-AAATGCACCATTTCCTGAGA-3′ |
| | | (SEQ ID NO: 4) |
| Oct4 | Foward | 5′-AGCAAAACCCGGAGGAGT-3′ |
| | | (SEQ ID NO: 5) |
| | Reverse | 5′-CCACATCGGCCTGTGTATATC-3′ |
| | | (SEQ ID NO: 6) |
| c-myc | Foward | 5′-AATGAAAAGGCCCCCAAGGT |
| | | AGTTATCC-3′ |
| | | (SEQ ID NO: 7) |
| | Reverse | 5′-AGCAAAACCCGGAGGAGT-3′ |
| | | (SEQ ID NO: 8) |
| Sox2 | Foward | 5′-TTGCTGCCTCTTTAAGACTAGGA-3′ |
| | | (SEQ ID NO: 9) |
| | Reverse | 5′-CTGGGGCTCAAACTTCTCTC-3′ |
| | | (SEQ ID NO: 10) |
| β-actin | Foward | 5′-TGTTACCAACTGGGACGACA-3′ |
| | | (SEQ ID NO: 11) |
| | Reverse | 5′-GGGGTGTTGAAGGTCTCAAA-3′ |
| | | (SEQ ID NO: 12) |

### <Experimental Example 11> Statistical Processing

All data were analyzed using GraphPad Prism 7.0 software (GraphPad Prism, Inc., San Diego, CA, USA). All data from three independent experiments were presented as mean±standard deviation (SD). Differences between means were analyzed using one-way ANOVA and Student's t-test. P-value values of less than 0.05 were considered significant.

### <Example 1> Isolation of Material of Inhibiting Breast Cancer Stem Cell Formation

Preparation of Dendropanax morbiferus Extract

Dried Dendropanax morbiferus H.Lev. leaves were obtained from Seogwipo-si, Jeju-do and a sample (no. 2020_011) was deposited at the Faculty of Biotechnology, Jeju National University (Jeju-Si, Korea). Dendropanax morbiferus H.Lev leaves were washed with water, freeze-dried, and crushed to prepare a total of 1 kg of Dendropanax morbiferus leaf powder. Thereafter, Dendropanax morbiferus leaf powder (40 g) and 1.2 L of 100% methanol were put to 25 3 L conical flasks and incubated at 30° C. overnight using a shaking incubator to prepare a Dendropanax morbiferus methanol extract.

Isolation of Compounds

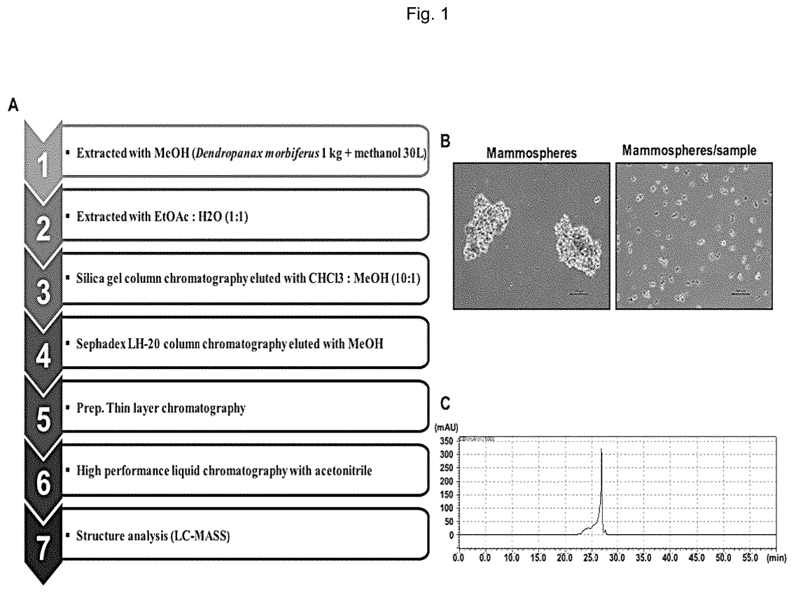

Candidate compounds having breast cancer stem cell formation inhibitory activity were isolated from Dendropanax morbiferus with reference to FIG. 1.

First, the methanol extract of Dendropanax morbiferus H. Lev prepared in Example 1-1 was filtered with filter paper (ADVANTEC®, Niigata, Japan), evaporated from 30 L to 5 L for 10 hours using a rotary evaporator (Heidolph, Schwabach, Germany), and added and mixed with a 2-fold volume of water (v/v=1:2) to evaporate methanol from the mixture at 55° C. The Dendropanax morbiferus extract from which methanol was removed was extracted with an equal volume of ethyl acetate (EA, v/v=1:1) using a separatory funnel (Sigma-Aldrich, Burlington, MA, USA) to obtain an ethyl acetate fraction.

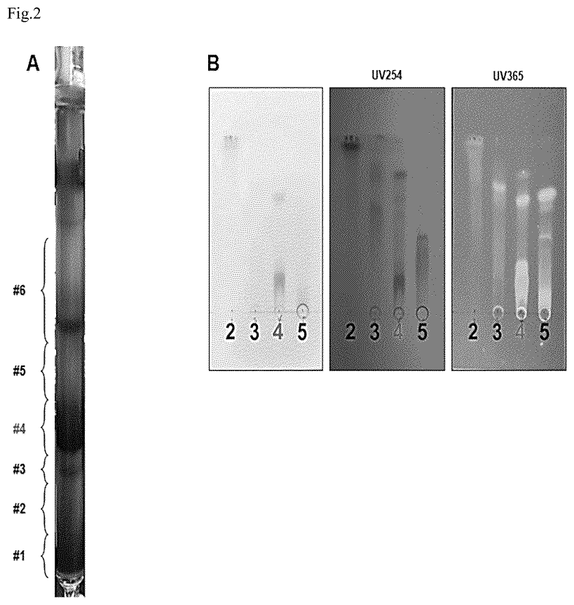

Thereafter, for chromatography, ethyl acetate was evaporated at 55° C. for 30 minutes using a rotary evaporator, and then concentrated with a chloroform-methanol solvent (CHCl₃:MeOH, 10:1). The concentrate was separated on a silica gel column (3×35 cm, 40 to 63 micron particle size) and eluted with a chloroform-methanol mixture (CHCl₃:MeOH, 10:1). The samples were separated about 20 times, and the column was fractionated into 6 parts on the basis of color (A of FIG. 2). Each fractionated sample was evaporated at 55° C. and dissolved in methanol, and then mammosphere formation analysis and TLC plate analysis were performed on 6 samples. As a result, it was found that Fraction #4 strongly inhibited the mammosphere formation in a breast cancer cell MCF-7 (B of FIG. 1), and as a result of TLC plate analysis, it was found that Fraction #4 was an active fraction (B of FIG. 2).

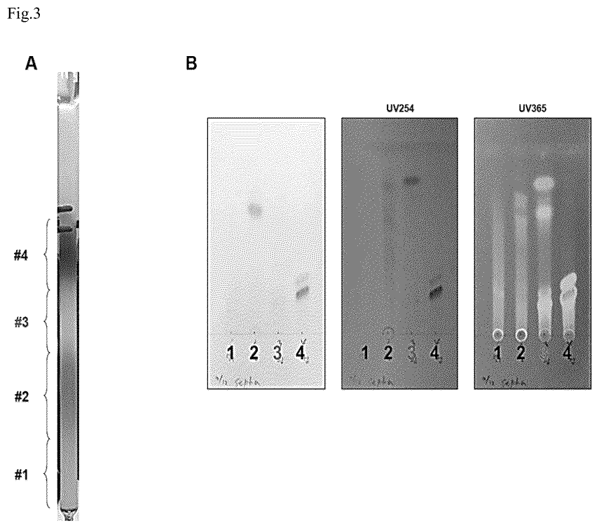

As Fraction #4, which had excellent mammosphere formation inhibitory ability, four fractions were obtained by using a Sephadex LH-20 gel column (2.5×30 cm, 25 to 100 micron article size) with methanol (A of FIG. 3). As a result of TLC plate analysis of the four fractions obtained by LH-20 gel column chromatography, Fraction #3 was found to be an active fraction (B of FIG. 3).

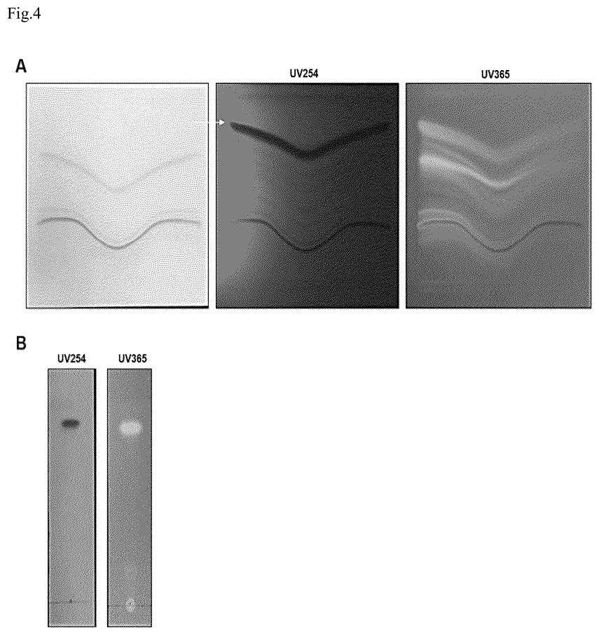

Subsequently, TLC chromatography (thin layer chromatography) was performed on the four fractions purified from Fraction #4 through a Sephadex LH-20 gel column. Specifically, the fractions were loaded on a preparatory TLC plate (prep-TLC) (glass plate; 20×20 cm) and loaded on a TLC glass chamber (CHCl₃:MeOH, 30:1) and developed. Thereafter, the plate was removed and dried, and then UV radiation (UV254 nm and UV365 nm) was irradiated to visualize fluorescence (FIG. 4). As a result, one band was observed as shown in B of FIG. 4. The band was separated from the plate, dissolved in methanol, centrifuged for 5 minutes, concentrated with methanol, and used for mammosphere formation analysis.

In the case of HPLC, the active band was analyzed on an HPLC instrument (Shimadzu LC-20A, Kyoto, Japan) equipped with an ODS column (10×250-mm, flow rate: 2 ml/min, mobile phase: acetonitrile-water). The acetonitrile concentration was initiated at 0%, increased to 60% at 10 minutes, reached 100% at 30 minutes, and eluted for 10 minutes. The HPLC results were illustrated in C of FIG. 1.

### <Example 2> Structural Analysis of Isolated Compound

The chemical structure of the isolated compound was analyzed via mass spectrometry and NMR.

As a result, the molecular weight was determined to be 358 by ESI-mass measurement, which showed quasi-molecular ion peaks at m/z 359.4 [M+H]⁺ and 381.3 [M+Na]⁺ in positive mode, and m/z 357.3 [M−H]⁻ in negative mode.

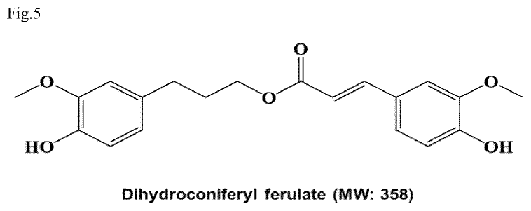

By confirming the structure of the compound through NMR, the compound having breast cancer stem cell inhibitory activity derived from leaves of Dendropanax morbiferus was identified as dihydroconiferyl ferulate (FIG. 5).

### <Example 3> Confirmation of Inhibitory Effect on Breast Cancer Stem Cell Formation of Dihydroconiferyl Ferulate

3-1. Confirmation of Breast Cancer Proliferation Inhibitory Effect

Breast cancer cells MCF-7 and MDA-MB-231 were treated with dihydroconiferyl ferulate, a compound purified from the leaves of Dendropanax morbiferus H.Lev at various concentrations to test an antiproliferative effect, and an experimental method was described in Experimental Example 3.

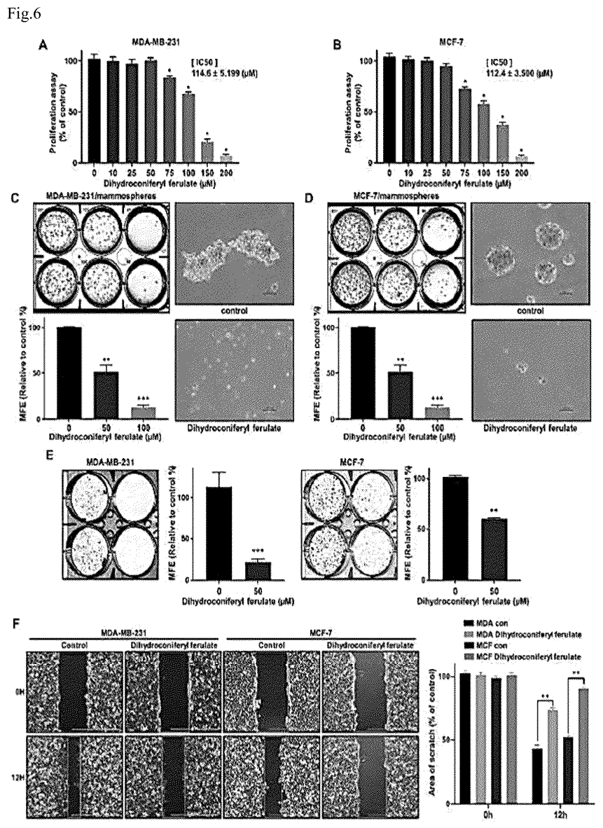

As a result, the dihydroconiferyl ferulate inhibited the proliferation of breast cancer cells at a concentration of 75 μM, and IC₅₀ values, which were the drug concentrations required for 50% growth reduction in a survival curve, were 112.4 μM and 114.6 μM (A and B of FIG. 6). These results indicate that dihydroconiferyl ferulate inhibits breast cancer cell proliferation.

3-2. Confirmation of Mammosphere Formation Inhibitory Effect of Breast Cancer Cells

Mammospheres formed from breast cancer cells MCF-7 and MDA-MB-231 were treated with dihydroconiferyl ferulate at a concentration of 50 μM, and a mammosphere formation assay was performed by automatic counting using the NICE program, and the experimental method was described in Experimental Example 3.

As a result, the dihydroconiferyl ferulate reduced the size and number of tumorspheres derived from MDA-MB-231 and MCF-7 cells (C and D of FIG. 6). These results indicate that dihydroconiferyl ferulate inhibits the mammosphere formation from breast cancer cells.

3-3. Confirmation of Breast Cancer Cell Colony Formation and Cell Migration Inhibitory Effect

Colony formation and cell migration assays were performed, and an experimental method was described in Experimental Example 4.

As a result, dihydroconiferyl ferulate inhibited colony formation and cell migration of MDA-MB-231 and MCF-7 cells (E and F of FIG. 6).

Since the cell colony formation and cell migration are likely to be two important processes in breast cancer tumorigenesis and metastasis, these results indicate that dihydroconiferyl ferulate is a potent suppressor of breast cancer cell colony formation and cell migration.

### <Example 4> Confirmation of Inhibitory Effect of Dihydroconiferyl Ferulate on CD44⁺/CD24⁻ Breast Cancer Cell Population

CD44⁺/CD24⁻, a representative marker of breast cancer stem cells, was associated with stem cell-like activity in breast cancer, and since CD44⁺/CD24⁻ MDA-MB-231 cells show higher tumorigenesis and metastasis than CD44−/CD24⁺ MDA-MB-231 cells, MDA-MB-231 cells were treated with dihydroconiferyl ferulate, and then changes in the CD44^{high}/CD24^{low} population were measured, and the experimental method was described in Experimental Example 5.

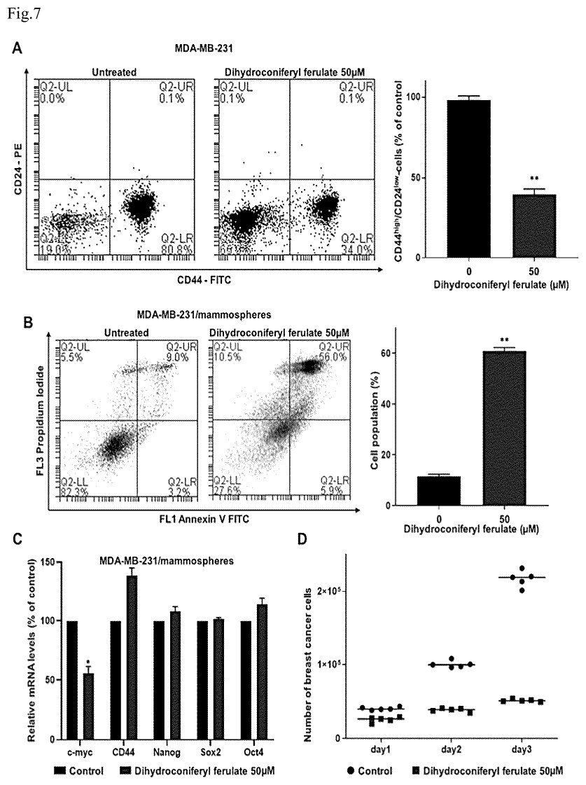

As a result, in MDA-MB-231 breast cancer cells, the CD44^{high}/CD24^{low} population decreased from 80.8% to 34.0% by dihydroconiferyl ferulate treatment (A of FIG. 7).

### <Example 5> Confirmation of Breast Cancer Stem Cell Growth Inhibition and Apoptosis Inducing Effect of Dihydroconiferyl Ferulate

5-1. Confirmation of Breast Cancer Stem Cell Apoptosis Effect

In order to confirm whether dihydroconiferyl ferulate induced apoptosis of breast cancer stem cells, mammospheres cultured for 5 days were treated with or without dihydroconiferyl ferulate (50 μM), and then an apoptosis assay was performed, and the experimental method was described in Experimental Example 6.

As a result, it was found that dihydroconiferyl ferulate induced the apoptosis of breast cancer stem cells (B of FIG. 7).

5-2. Confirmation of Breast Cancer Stem Cell Growth Inhibitory Effect

To determine whether dihydroconiferyl ferulate inhibited the growth of breast cancer stem cells, MDA-MB-231-derived mammospheres were treated with dihydroconiferyl ferulate (50 μM), and the mammospheres were separated into single cells, dispensed in a 6-well plate, and the number of cells was counted for 3 days. In addition, qRT-PCR was performed to confirm transcription levels of cancer stem cell-specific markers c-myc, CD44, Nanog, Sox2, and Oct4 to confirm changes in mRNA levels. The experimental method was described in Experimental Example 9.

As a result, it was found that dihydroconiferyl ferulate reduced c-Myc gene expression (C of FIG. 7), and reduced the cell count of mammospheres and inhibited the mammosphere formation (D of FIG. 7).

### <Example 6> Confirmation of Cancer Stem Cell Inhibition Mechanism of Dihydroconiferyl Ferulate

6-1. Confirmation of EGFR Signaling Inhibitory Effect in Breast Cancer Stem Cells

In order to identify the underlying molecular mechanism associated with the inhibition of mammosphere formation by dihydroconiferyl ferulate, total and nuclear protein levels of EGFR were evaluated through Western blot analysis, and the experimental method was described in Experimental Example 7.

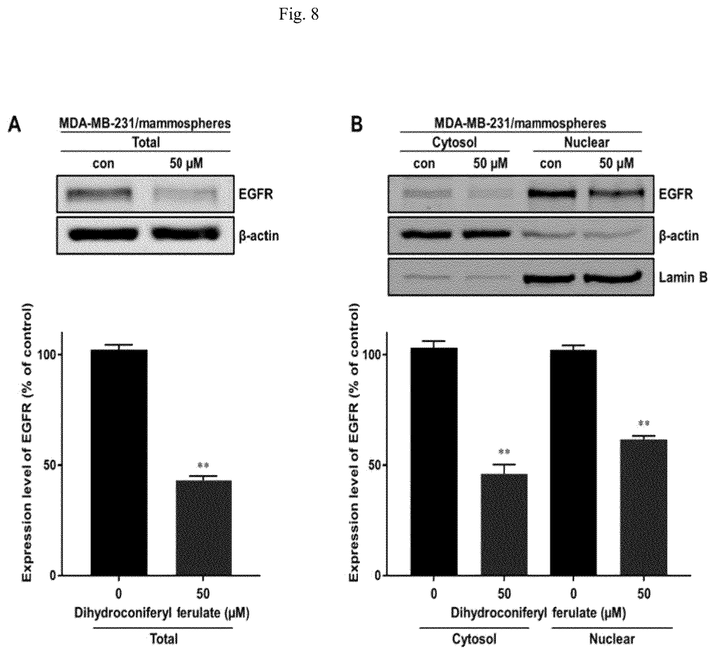

As a result, the total and nuclear protein levels of EGFR were significantly decreased after 48 hours of treatment with dihydroconiferyl ferulate (50 μM) (FIG. 8).

Breast cancer cells overexpressed the EGFR protein and the EGFR exhibited membrane-bound and nuclear signaling activities. Nuclear EGFR was a key therapeutic target for breast cancer by inducing resistance to anti-EGFR therapy. Nuclear EGER (nEGFR) is also a regulator and cofactor of Stat3.

Therefore, an immunoprecipitation assay was performed to evaluate the interaction between Stat3 and EGFR proteins, and Western blot analysis was performed to evaluate total protein, cytosolic protein and nuclear protein levels of p-Stat3 and Stat3. Experimental methods were described in Experimental Example 7 and Experimental Example 8.

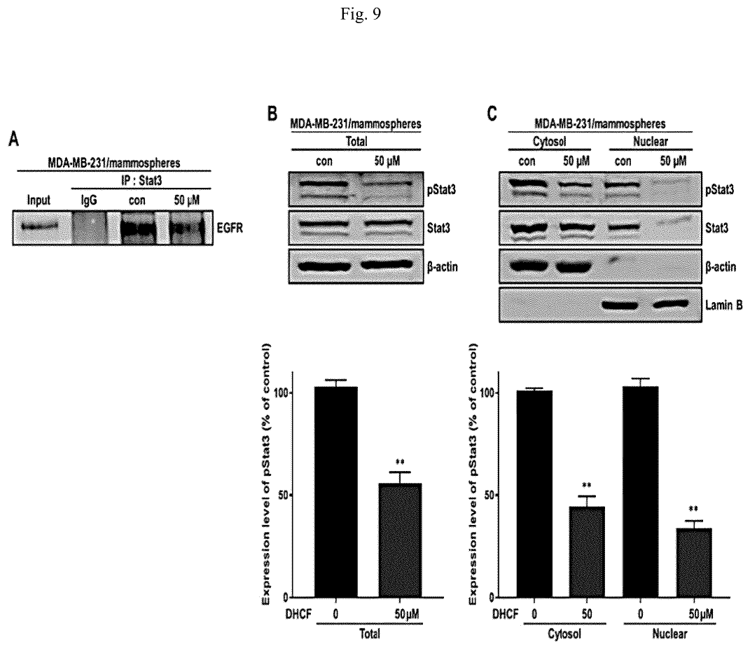

As a result, it was shown that dihydroconiferyl ferulate inhibited the interaction between EGFR and Stat3 (A of FIG. 9). In addition, not only the levels of p-Stat3 and Stat3 in the nucleus and cytoplasm of mammospheres were reduced by treatment with dihydroconiferyl ferulate, but also the total level of p-Stat3 was decreased (B and C of FIG. 9). 6-2. c-Myc inhibition of dihydroconiferyl ferulate

Nuclear EGFR was known to function as a co-transcription factor for Stat3 to enhance the transcription of a c-myc gene. Therefore, mRNA and total protein, cytosolic protein, and nuclear protein levels of c-Myc and Stat3 were determined to examine the inhibitory effect on c-Myc transcription through an EGFR-Stat3 complex, which was inhibited by treatment of dihydroconiferyl ferulate.

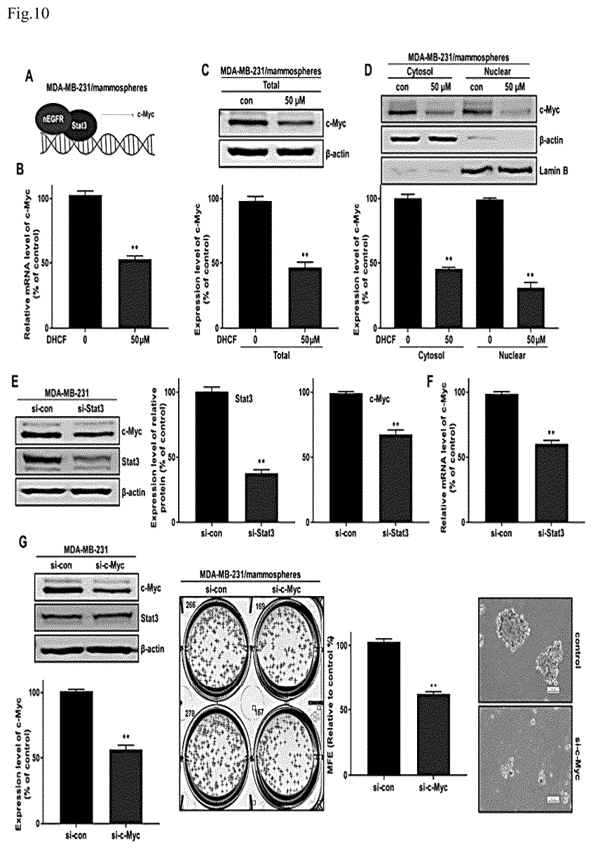

As a result, it was found that the c-Myc mRNA level was reduced by dihydroconiferyl ferulate (A and B of FIG. 10), and the total and nuclear levels of the c-Myc protein were also decreased (C and D of FIG. 10). In addition, the downregulation of Stat3 decreased the protein and mRNA expression levels of c-Myc (E and F of FIG. 10), and knockout of c-Myc inhibited the mammosphere formation (G of FIG. 10). These results suggest that dihydroconiferyl ferulate inhibits c-Myc expression via Stat3 and/or EGFR-Stat3 complex inhibition.

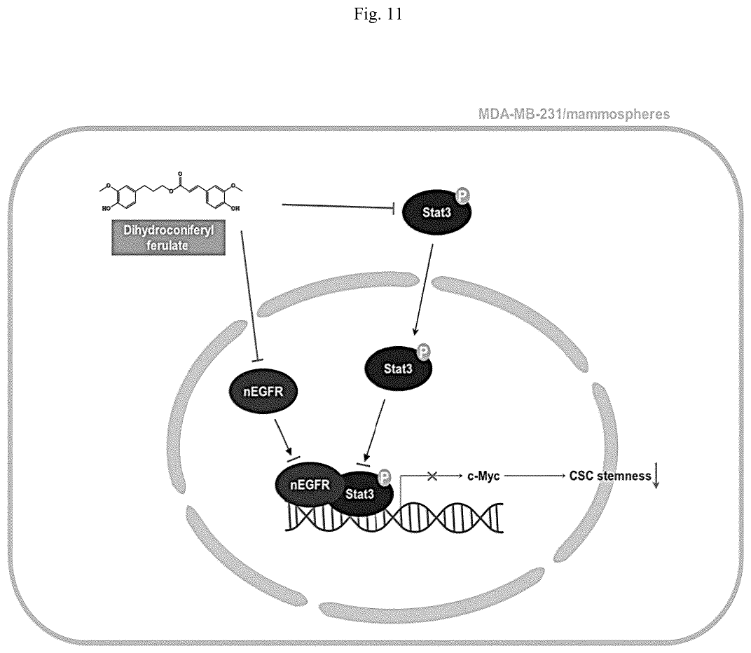

FIG. 11 is a schematic diagram briefly showing a mechanism through which dihydroconiferyl ferulate inhibits the formation of breast cancer stem cells via an EGFR-Stat3/c-Myc signaling pathway.

As described above, the specific embodiments of the present disclosure have been described, but those skilled in the art understanding the spirit of the present disclosure will be able to easily propose other degenerate inventions or other embodiments included in the scope of the present disclosure by adding, changing, and deleting other elements within the same technical scope. Therefore, it should be appreciated that the embodiments described above are illustrative in all aspects and are not restricted. The scope of the present disclosure is represented by claims to be described below rather than the detailed description, and it is to be interpreted that the meaning and scope of the claims and all the changes or modified forms derived from the equivalents thereof come within the scope of the present disclosure.

From the foregoing, it will be appreciated that various embodiments of the present disclosure have been described herein for purposes of illustration, and that various modifications may be made without departing from the scope and spirit of the present disclosure. Accordingly, the various embodiments disclosed herein are not intended to be limiting, with the true scope and spirit being indicated by the following claims.

What is claimed is:

1. A method for treating breast cancer, comprising administering a composition comprising a Dendropanax morbiferus extract or a fraction thereof as an active ingredient to a subject in need thereof.

2. The method of claim 1, wherein the extract is extracted from leaves of Dendropanax morbiferus.

3. The method of claim 1, wherein the extract is extracted with one or more solvents selected from the group consisting of water, organic solvents, subcritical fluids and supercritical fluids.

4. The method of claim 1, wherein the fraction is an ethanol fraction, a methanol fraction, a dichloromethane fraction, an ethyl acetate fraction, a water fraction, an n-hexane fraction, a chloroform fraction, or a butanol fraction.

5. The method of claim 1, wherein the composition inhibits growth of breast cancer stem cells.

6. The method of claim 1, wherein the composition inhibits formation of breast cancer-derived mammospheres or inhibits proliferation of breast cancer-derived mammospheres.

7. The method of claim 1, wherein the composition inhibits expression of a c-Myc gene or protein.

8. The method of claim 1, wherein the breast cancer is breast cancer expressing CD44^{high}/CD24^{low}.

9. A method for treating breast cancer, comprising administering a composition comprising dihydroconiferyl ferulate or a pharmaceutically acceptable salt thereof as an active ingredient to a subject in need thereof, wherein the dihydroconiferyl ferulate is represented by Chemical Formula 1 below:

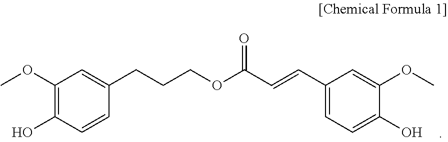

10. The method of claim 9, wherein the composition inhibits growth of breast cancer stem cells.

11. A method for treating breast cancer, comprising administering i) a composition comprising dihydroconiferyl ferulate or a pharmaceutically acceptable salt thereof as an active ingredient to a subject in need thereof and ii) a anticancer agent, wherein the dihydroconiferyl ferulate is represented by Chemical Formula 1 below:

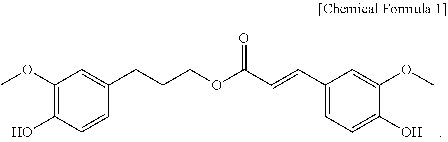

12. The method of claim 11, wherein the anticancer agent is eribulin, carboplatin, cisplatin, Halaven, 5-fluorouracil (5-FU), gleevec, Vincristine, Vinblastine, Vinorelvine, Paclitaxel, Docetaxel, Etoposide, Topotecan, Irinotecan, Dactinomycin, Doxorubicin, Daunorubicin, valrubicin, flutamide, gemcitabine, Mitomycin, or Bleomycin.

13. The method of claim 11, wherein the composition increases apoptosis of cancer cells, multidrug-resistant cancer cells or cancer stem cells.


SEQUENCE LISTING (12 sequences)

SEQ ID NO: 1 - DNA, 20 bp, synthetic construct
  source 1..20: mol_type = other DNA
atgcctcacacggagactgt

SEQ ID NO: 2 - DNA, 20 bp, synthetic construct
  source 1..20: mol_type = other DNA
aagtgggttgtttgcctttg

SEQ ID NO: 3 - DNA, 20 bp, synthetic construct
  source 1..20: mol_type = other DNA
agaaggtgtgggcagaagaa

SEQ ID NO: 4 - DNA, 20 bp, synthetic construct
  source 1..20: mol_type = other DNA
aaatgcaccatttcctgaga

SEQ ID NO: 5 - DNA, 18 bp, synthetic construct
  source 1..18: mol_type = other DNA
agcaaaacccggaggagt

SEQ ID NO: 6 - DNA, 21 bp, synthetic construct
  source 1..21: mol_type = other DNA
ccacatcggcctgtgtatatc

SEQ ID NO: 7 - DNA, 28 bp, synthetic construct
  source 1..28: mol_type = other DNA
aatgaaaaggcccccaaggtagttatcc

SEQ ID NO: 8 - DNA, 18 bp, synthetic construct
  source 1..18: mol_type = other DNA
agcaaaacccggaggagt

SEQ ID NO: 9 - DNA, 23 bp, synthetic construct
  source 1..23: mol_type = other DNA
ttgctgcctctttaagactagga

SEQ ID NO: 10 - DNA, 20 bp, synthetic construct
  source 1..20: mol_type = other DNA
ctggggctcaaacttctctc

SEQ ID NO: 11 - DNA, 20 bp, synthetic construct
  source 1..20: mol_type = other DNA
tgttaccaactgggacgaca

SEQ ID NO: 12 - DNA, 20 bp, synthetic construct
  source 1..20: mol_type = other DNA
ggggtgttgaaggtctcaaa